SEQUENCE LISTING (110 sequences)

SEQ ID NO: 1 - DNA, 22 bp, Artificial Sequence
  PCR Primer
cagctatccacccacacagatg

SEQ ID NO: 2 - DNA, 22 bp, Artificial Sequence
  PCR Primer
cgaaggctccaaagaagacagt

SEQ ID NO: 3 - DNA, 20 bp, Artificial Sequence
  PCR Primer
tcaatcggcccgactatctc

SEQ ID NO: 4 - DNA, 20 bp, Artificial Sequence
  PCR Primer
cagggcaatgatcccaaagt

SEQ ID NO: 5 - DNA, 23 bp, Artificial Sequence
  PCR Primer
agggtgtacgtcaacatcagtca

SEQ ID NO: 6 - DNA, 18 bp, Artificial Sequence
  PCR Primer
cacggccccaaagaaggt

SEQ ID NO: 7 - DNA, 21 bp, Artificial Sequence
  PCR Primer
gcccctcttccaactgaagaa

SEQ ID NO: 8 - DNA, 25 bp, Artificial Sequence
  PCR Primer
ggtattgtcagtggtcacattcaag

SEQ ID NO: 9 - DNA, 19 bp, Artificial Sequence
  PCR Primer
ccacagttccgccaaacct

SEQ ID NO: 10 - DNA, 24 bp, Artificial Sequence
  PCR Primer
cacctggttgcaattcaaatactc

SEQ ID NO: 11 - DNA, 24 bp, Artificial Sequence
  PCR Primer
tggcagcatcttcacttctaaatg

SEQ ID NO: 12 - DNA, 24 bp, Artificial Sequence
  PCR Primer
gaaatgagtccccaaaacatctct

SEQ ID NO: 13 - DNA, 22 bp, Artificial Sequence
  PCR Primer
cacactctgcaccaacctcact

SEQ ID NO: 14 - DNA, 23 bp, Artificial Sequence
  PCR Primer
tgcactccaaagaaggtctcatc

SEQ ID NO: 15 - DNA, 24 bp, Artificial Sequence
  PCR Primer
accaccatatcagtcaatgtggat

SEQ ID NO: 16 - DNA, 25 bp, Artificial Sequence
  PCR Primer
gaagatggacaacacattctcaaga

SEQ ID NO: 17 - DNA, 23 bp, Artificial Sequence
  PCR Primer
agctacaaagaggacacgaagga

SEQ ID NO: 18 - DNA, 24 bp, -Artificial Sequence
  PCR Primer
cgcagctctagttgaaagaagaca

SEQ ID NO: 19 - DNA, 26 bp, Artificial Sequence
  PCR Primer
gggaatatttgagcttaaggaaaatg

SEQ ID NO: 20 - DNA, 20 bp, Artificial Sequence
  PCR Primer
aaaaggccccgaaaaaactg

SEQ ID NO: 21 - DNA, 30 bp, Artificial Sequence
  PCR Primer
gagtatcttcaactaatggtgtacgtcact

SEQ ID NO: 22 - DNA, 21 bp, Artificial Sequence
  PCR Primer
tggtgcttcctcctttcatca

SEQ ID NO: 23 - DNA, 24 bp, Artificial Sequence
  PCR Primer
tactgtcaggtgcactttgatgag

SEQ ID NO: 24 - DNA, 19 bp, Artificial Sequence
  PCR Primer
gcagtggctgagaattcct

SEQ ID NO: 25 - DNA, 22 bp, Artificial Sequence
  PCR Primer
atatggtgtccgaatccaggat

SEQ ID NO: 26 - DNA, 21 bp, Artificial Sequence
  PCR Primer
cctgacccatggtgaaagtca

SEQ ID NO: 27 - DNA, 25 bp, Artificial Sequence
  PCR Primer
atgcctgaaacactacccaataatt

SEQ ID NO: 28 - DNA, 24 bp, Artificial Sequence
  PCR Primer
gcaagttggagttcatctccttct

SEQ ID NO: 29 - DNA, 18 bp, Artificial Sequence
  PCR Primer
cgtccgtgtgctggatga

SEQ ID NO: 30 - DNA, 22 bp, Artificial Sequence
  PCR Primer
catgaaagccccgaagtaagac

SEQ ID NO: 31 - DNA, 23 bp, Artificial Sequence
  PCR Primer
tgcgaagtaggtagcaactggtt

SEQ ID NO: 32 - DNA, 22 bp, Artificial Sequence
  PCR Primer
ccattagcttgtccccttcttg

SEQ ID NO: 33 - DNA, 26 bp, Artificial Sequence
  PCR Primer
cggctgtataaaaacaaagacatgat

SEQ ID NO: 34 - DNA, 22 bp, Artificial Sequence
  PCR Primer
tccccaacatgcaattcataag

SEQ ID NO: 35 - DNA, 24 bp, Artificial Sequence
  PCR Primer
gaagatggaaaagcgatttgtctt

SEQ ID NO: 36 - DNA, 21 bp, Artificial Sequence
  PCR Primer
gggcatgttttctgcttgaga

SEQ ID NO: 37 - DNA, 20 bp, Artificial Sequence
  PCR Primer
gctcttggagctgcctacgt

SEQ ID NO: 38 - DNA, 24 bp, Artificial Sequence
  PCR Primer
aaggtctctttcaccaatgcactt

SEQ ID NO: 39 - DNA, 24 bp, Artificial Sequence
  PCR Primer
tcatcactggtcttttggagtttg

SEQ ID NO: 40 - DNA, 20 bp, Artificial Sequence
  PCR Primer
tctgcacagctctggcttgt

SEQ ID NO: 41 - DNA, 27 bp, Artificial Sequence
  PCR Primer
tgctaaagaacttagatgtcagtgcat

SEQ ID NO: 42 - DNA, 23 bp, Artificial Sequence
  PCR Primer
tggtccactctcaatcactctca

SEQ ID NO: 43 - DNA, 20 bp, Artificial Sequence
  PCR Primer
gcaggccctgaatttcaaca

SEQ ID NO: 44 - DNA, 29 bp, Artificial Sequence
  PCR Primer
gaagtatgcagagcttgattttagtttta

SEQ ID NO: 45 - DNA, 29 bp, Artificial Sequence
  PCR Primer
gaagtatgcagagcttgattttagtttta

SEQ ID NO: 46 - DNA, 20 bp, Artificial Sequence
  PCR Primer
cccattcgctccaagatgag

SEQ ID NO: 47 - DNA, 25 bp, Artificial Sequence
  PCR Primer
tgaagtgctttctcttggagttaca

SEQ ID NO: 48 - DNA, 26 bp, Artificial Sequence
  PCR Primer
cattcccattagaagacaaactgttg

SEQ ID NO: 49 - DNA, 22 bp, Artificial Sequence
  PCR Primer
aaaacctcttgggaagcatgag

SEQ ID NO: 50 - DNA, 20 bp, Artificial Sequence
  PCR Primer
gggaccccgaggacagtact

SEQ ID NO: 51 - DNA, 20 bp, Artificial Sequence
  PCR Primer
ccattgtggccaaggagatc

SEQ ID NO: 52 - DNA, 19 bp, Artificial Sequence
  PCR Primer
tgtccaggtggtccatgga

SEQ ID NO: 53 - DNA, 28 bp, Artificial Sequence
  PCR Primer
cacagaatttcatagctgactactttga

SEQ ID NO: 54 - DNA, 22 bp, Artificial Sequence
  PCR Primer
tcgcttggttaggaagatgaca

SEQ ID NO: 55 - DNA, 23 bp, Artificial Sequence
  PCR Primer
ggtattccaaaccaaaagaagca

SEQ ID NO: 56 - DNA, 26 bp, Artificial Sequence
  PCR Primer
gttcagttccaggtcatacacgtact

SEQ ID NO: 57 - DNA, 21 bp, Artificial Sequence
  PCR Primer
agtcgtctttgtcacccgaaa

SEQ ID NO: 58 - DNA, 26 bp, Artificial Sequence
  PCR Primer
agctcatctccaaagagttgatgtac

SEQ ID NO: 59 - DNA, 19 bp, Artificial Sequence
  PCR Primer
tgtgctgaccccacacaga

SEQ ID NO: 60 - DNA, 23 bp, Artificial Sequence
  PCR Primer
gctttggagtttgggttttcttg

SEQ ID NO: 61 - DNA, 26 bp, Artificial Sequence
  PCR Primer
agagctacacaagaatcaccaacatc

SEQ ID NO: 62 - DNA, 19 bp, Artificial Sequence
  PCR Primer
agacctccttgccccgttt

SEQ ID NO: 63 - DNA, 22 bp, Artificial Sequence
  PCR Primer
cccagaaagctgtgatcttcaa

SEQ ID NO: 64 - DNA, 21 bp, Artificial Sequence
  PCR Primer
tcctgcacccacttcttcttg

SEQ ID NO: 65 - DNA, 21 bp, Artificial Sequence
  PCR Primer
ccaaactgggcaaggagatct

SEQ ID NO: 66 - DNA, 24 bp, Artificial Sequence
  PCR Primer
ggcccaggtgtttcatataattct

SEQ ID NO: 67 - DNA, 27 bp, Artificial Sequence
  PCR Primer
tgcttcacctacactacctacaagatc

SEQ ID NO: 68 - DNA, 18 bp, Artificial Sequence
  PCR Primer
gacaattccgggcttgga

SEQ ID NO: 69 - DNA, 27 bp, Artificial Sequence
  PCR Primer
cagattccacaaaagttcatagttgac

SEQ ID NO: 70 - DNA, 19 bp, Artificial Sequence
  PCR Primer
ccggcctctcttggttagg

SEQ ID NO: 71 - DNA, 24 bp, Artificial Sequence
  PCR Primer
ctgctgtagtgttcaccacactga

SEQ ID NO: 72 - DNA, 24 bp, Artificial Sequence
  PCR Primer
ctgctgtagtgttcaccacactga

SEQ ID NO: 73 - DNA, 28 bp, Artificial Sequence
  PCR Primer
gatacacagaccgtattcttcatcctaa

SEQ ID NO: 74 - DNA, 26 bp, Artificial Sequence
  PCR Primer
tgaaagatgatagcattgatgtcaca

SEQ ID NO: 75 - DNA, 21 bp, Artificial Sequence
  PCR Primer
cgctctcaggcagagctatgt

SEQ ID NO: 76 - DNA, 21 bp, Artificial Sequence
  PCR Primer
cttgtccagatgctgcatcag

SEQ ID NO: 77 - DNA, 19 bp, Artificial Sequence
  PCR Primer
gcgcgtggtgaaacacttc

SEQ ID NO: 78 - DNA, 22 bp, Artificial Sequence
  PCR Primer
atcggcacagatctccttatcc

SEQ ID NO: 79 - DNA, 20 bp, Artificial Sequence
  PCR Primer
cgaagcatcccgtgttcact

SEQ ID NO: 80 - DNA, 18 bp, Artificial Sequence
  PCR Primer
gatgacacccggcttgga

SEQ ID NO: 81 - DNA, 22 bp, Artificial Sequence
  PCR Primer
caggagtgatcttcaccaccaa

SEQ ID NO: 82 - DNA, 20 bp, Artificial Sequence
  PCR Primer
ggcgtccaggttcttcatgt

SEQ ID NO: 83 - DNA, 20 bp, Artificial Sequence
  PCR Primer
ggcgtccaggttcttcatgt

SEQ ID NO: 84 - DNA, 23 bp, Artificial Sequence
  PCR Primer
gtagaatatcgcagcaggcagat

SEQ ID NO: 85 - DNA, 22 bp, Artificial Sequence
  PCR Primer
ctgcttccaatacagccacaag

SEQ ID NO: 86 - DNA, 24 bp, Artificial Sequence
  PCR Primer
gagcagctgttactggtgaattca

SEQ ID NO: 87 - DNA, 19 bp, Artificial Sequence
  PCR Primer
cgtgcttcacctggctcaa

SEQ ID NO: 88 - DNA, 21 bp, Artificial Sequence
  PCR Primer
ggtgctcaaaccactgtgaca

SEQ ID NO: 89 - DNA, 22 bp, Artificial Sequence
  PCR Primer
ggaaatgtttgccacaggaaga

SEQ ID NO: 90 - DNA, 21 bp, Artificial Sequence
  PCR Primer
tgtttcgtgtttcccctgatg

SEQ ID NO: 91 - DNA, 16 bp, Artificial Sequence
  PCR Primer
ccactgcgcccaaacc

SEQ ID NO: 92 - DNA, 20 bp, Artificial Sequence
  PCR Primer
gcaggattgaggcaagcttt

SEQ ID NO: 93 - DNA, 18 bp, Artificial Sequence
  PCR Primer
cccctggccactgaactg

SEQ ID NO: 94 - DNA, 23 bp, Artificial Sequence
  PCR Primer
tggatgttcttgaggtgaattcc

SEQ ID NO: 95 - DNA, 23 bp, Artificial Sequence
  PCR Primer
ggaattcacctcaagaacatcca

SEQ ID NO: 96 - DNA, 21 bp, Artificial Sequence
  PCR Primer
gtggctatgacttcggtttgg

SEQ ID NO: 97 - DNA, 18 bp, Artificial Sequence
  PCR Primer
ccgtcccaggcacatcac

SEQ ID NO: 98 - DNA, 18 bp, Artificial Sequence
  PCR Primer
ccgtcccaggcacatcac

SEQ ID NO: 99 - DNA, 19 bp, Artificial Sequence
  PCR Primer
agagctgcgttgcgtttgt

SEQ ID NO: 100 - DNA, 22 bp, Artificial Sequence
  PCR Primer
tggcgaacacttgcagattact

SEQ ID NO: 101 - DNA, 20 bp, Artificial Sequence
  PCR Primer
cagagctgcgttgcacttgt

SEQ ID NO: 102 - DNA, 23 bp, Artificial Sequence
  PCR Primer
acacctgcagtttaccaatcgtt

SEQ ID NO: 103 - DNA, 22 bp, Artificial Sequence
  PCR Primer
tctggaattcatcccaaaaaca

SEQ ID NO: 104 - DNA, 22 bp, Artificial Sequence
  PCR Primer
tctggaattcatcccaaaaaca

SEQ ID NO: 105 - DNA, 25 bp, Artificial Sequence
  PCR Primer
ccacctacaatccttgaaagacctt

SEQ ID NO: 106 - DNA, 27 bp, Artificial Sequence
  PCR Primer
cagtgtagcaatgatttcaattttctc

SEQ ID NO: 107 - DNA, 21 bp, Artificial Sequence
  PCR Primer
tccacgtgttgagatcattgc

SEQ ID NO: 108 - DNA, 21 bp, Artificial Sequence
  PCR Primer
tcttgatggccttcgattctg

SEQ ID NO: 109 - DNA, 19 bp, Artificial Sequence
  PCR Primer
cccacagccaggacatcag

SEQ ID NO: 110 - DNA, 22 bp, Artificial Sequence
  PCR Primer
cttgcacagcacataggaaagg

(19) United States
(12) Patent Application Publication
(10) Pub. No.: US 2009/0311684 A1
(43) Pub. Date: Dec. 17, 2009

(54) ENHANCED FC RECEPTOR-MEDIATED TUMOR NECROSIS FACTOR SUPERFAMILY AND CHEMOKINE MRNA EXPRESSION IN PERIPHERAL BLOOD LEUKOCYTES IN PATIENTS WITH RHEUMATOID ARTHRITIS

(75) Inventors: Masato Mitsuhashi, Irvine, CA (US); Katsuya Endo, Ibaraki (JP)

Correspondence Address: KNOBBE MARTENS OLSON & BEAR LLP, 2040 MAIN STREET, FOURTEENTH FLOOR, IRVINE, CA, 92614, US

(73) Assignees: Hitachi Chemical Co., Ltd, Shinjuku-ku Tokyo (JP); Hitachi Chemical research Center Inc.

(21) Appl. No.: 12/296,423

(22) PCT Filed: Apr. 5, 2007
(86) PCT No.: PCT/US07/08559

§ 371 Date: Oct. 7, 2008

Related U.S. Application Data
(60) Provisional application No. 60/790,511, filed on Apr. 7, 2006.

(51) Int. Cl. C12Q 1/68

(52) U.S. Cl. 435/6

(57) ABSTRACT

A method for predicting patient responsiveness to rheumatoid arthritis treatments involving altering expression of TNFSF-3, TNFSF-4, TNFSF-7, TNFSF-11, or TNFSF-14 is disclosed. A method for monitoring the effectiveness of such therapy is also disclosed. Furthermore, a method of screening compounds for use in the treatment of rheumatoid arthritis is disclosed. A method of monitoring the disease state over time in rheumatoid arthritis patients is also disclosed.

This application claims priority to U.S. Provisional Patent Application No. 60/790,511, filed Apr. 7, 2006, titled ENHANCED FC RECEPTOR-MEDIATED TNFSF mRNA EXPRESSION IN PERIPHERAL BLOOD LEUKOCYTES IN PATIENTS WITH RHEUMATOID ARTHRITIS AND COLLAGEN DISEASES, which is hereby incorporated by reference in its entirety and made part of this specification.

### BACKGROUND

1. Field

The disclosure relates to a method for predicting patient responsiveness to treatments for rheumatoid arthritis involving a tumor necrosis factor superfamily member or a cytokine, to a method of monitoring the effectiveness of such therapy, and to a method for screening compounds for use in the treatment of rheumatoid arthritis. The disclosure also relates to a method for monitoring the disease state in rheumatoid arthritis patients.

2. Description of the Related Art

Autoimmune disease is characterized by production of either antibodies that react with host cells or immune effector T cells that are autoreactive. Autoantibodies are frequently identified in certain types of autoimmune disease, such as anti-acetylcholine receptor antibodies in myasthenia gravis and anti-DNA antibodies in systemic lupus erythematosus. However, such autoantibodies are not seen in many types of autoimmune disease. Moreover, autoantibodies are often detected among healthy individuals, but such antibodies do not induce autoimmune disease. Thus, beside autoantibodies, additional yet-to-be identified mechanisms are evidently involved in the pathogenesis of autoimmune disease.

Once autoantibodies bind to the target host cells, the complement cascade is thought to be activated to form the C5-9 membrane attack complex on the target cell membranes, which leads to the death of host cells (see Esser, Toxicology 87, 229 (1994)). By-product chemotactic factors, such as C3a, C4a, or C5a recruit more leukocytes to the lesion (see Hugli, Crit. Rev. Immunol. 1, 321 (1981)). Recruited leukocytes or naturally present leukocytes at the lesion recognize antibody-bound cells (immune complex) via Fe receptors (“FcR”). Once the FcR is cross-bridged by the immune complex, leukocytes release TNF-ao (see Debets et al., J. Immunol. 141, 1197 (1988)), which binds to specific receptors present on the surface of host cells, and induce apoptosis or cell damage (see Micheau et al., Cell 114, 181 (2003)). Activated FcR also initiates the release of chemotactic cytokines to recruit different subsets of leukocytes to the lesion (see Chantry et al., Eur. J. Immunol. 19, 189 (1989)). This is an overall hypothesis of the molecular mechanism of FcR-related autoimmune disease.

Rheumatoid arthritis (“RA”) is an immune disease involving inflammation of the gastrointestinal tract. Although it is well characterized clinically, its pathogenesis is poorly understood. RA is characterized by persistent inflammatory synovitis, usually involving peripheral joints in a symmetric distribution. This may lead to cartilage destruction, bone erosion, and changes in joint integrity. The cause of RA remains unknown, but it is strongly suspected that CD4+ T-cells play a role in the disease because of the predominance of such cells in the synovium, the increase in soluble IL-2 receptors (produced by activated T cells) in the blood and serum of RA patients, and the noted amelioration of the disease by removal of T cells. Treatment focuses on pain relief, reduction of inflammation, protection of articular structures, maintenance of function, and control of systemic involvement. Options include: aspirin and other nonsteroidal anti-inflammatory drugs; antirheumatic drugs such as methotrexate, gold compounds, D-penicillamine, the antimalaraials, and sulfasalazine; glucocorticoids; TNF-alpha neutralizing agents such as infliximab and etanercept; and immunosuppressive drugs such as azathioprine, leflunomide, cyclosporine, and cyclophosphamide. Because the choice of therapeutic options depends on an assessment of the disease state in RA patients, it would be desirable to develop new methods of evaluating the disease state and monitoring the progression of the disease.

TNFSF11 (also known as TRANCE, CDF254, and RANK ligand) is a recently described member of the tumor necrosis factor superfamily which, when expressed by activated T cells, enhances the survival of antigen-presenting dendritic cells, and when expressed and secreted by osteoblasts, promotes the differentiation and activation of osteoclasts via a cell-surface receptor, TNFRSF11 (also known as RANK). Osteoclasts participate in bone resorption when activated, and may be in part responsible for the bone erosion observed in some RA patients.

### SUMMARY

In an embodiment, a method of determining whether a human having rheumatoid arthritis is likely to respond to a therapy is provided that comprises: stimulating leukocytes in vitro in a first sample that comprises leukocytes from the human; after the stimulation, measuring the amount of an mRNA selected from the group consisting of tumor necrosis factor subfamily (“TNFSF”)-3, TNFSF-4, TNFSF-7, TNFSF-11, and TNFSF-14 in the first sample; stimulating leukocytes in vitro in a second sample comprising leukocytes from the human with a control stimulus; measuring the amount of the mRNA in the second sample; and determining a ratio of the amount of the mRNA in the first sample to the amount of the mRNA in the second sample, wherein the human is likely to respond to the therapy if the ratio is about 1.5:1 or greater.

In a further aspect, stimulating leukocytes in the first sample comprises intermixing heat-aggregated human IgG with the first sample.

In a further aspect, stimulating leukocytes in the first sample comprises intermixing with the sample an agent selected from the group consisting of phorbol myristate acetate (PMA), phytohemagglutinin (PHA), wheat germ agglutinin (WGA), concanavalin-A (ConA), lipopolysaccharides (LPS), jacalin, fucoidan, heat-aggregated IgE, heat-aggregated IgA, and heat-aggregated IgM.

In a further aspect, at least one of the first and second samples comprises whole blood.

In another aspect, the control stimulus is phosphate buffered saline.

In a further aspect, the therapy comprises administration of an agent selected from the group consisting of cyclosporine A and tacrolimus.

In an embodiment, a method of evaluating the effectiveness of a therapy for rheumatoid arthritis is provided that comprises: stimulating leukocytes in vitro in a first sample comprising leukocytes from the human; stimulating leukocytes in vitro in a second sample comprising leukocytes from the human with a control stimulus; measuring the amount of an mRNA selected from the group consisting of tumor necrosis factor subfamily (“TNFSF”)-3, TNFSF-4, TNFSF-7, TNFSF-11, and TNFSF-14 in the first and second samples after stimulation; calculating a first ratio of the amount of the mRNA in the first sample to the amount of the mRNA in the second sample; administering the therapy to the human; stimulating leukocytes in vitro in a third sample comprising leukocytes from the human obtained after the administration of therapy; stimulating leukocytes in vitro in a fourth sample comprising leukocytes from the human obtained after the administration of therapy with the control stimulus; measuring the level of the mRNA in the third and fourth samples after stimulation; calculating a second ratio of the amount of the mRNA in the third sample to the amount of the mRNA in the fourth sample; and comparing the first and second ratios, wherein a significant difference in the ratios is indicative of an effective therapy.

In a further aspect, stimulating leukocytes in the first and third samples comprises intermixing heat-aggregated human IgG with the sample.

In a further aspect, stimulating leukocytes in the first and third samples comprises intermixing with the sample an agent selected from the group consisting of phorbol myristate acetate (PMA), phytohemagglutinin (PHA), wheat germ agglutinin (WGA), concanavalin-A (ConA), lipopolysaccharides (LPS), jacalin, fucoidan, heat-aggregated IgE, heat-aggregated IgA, and heat-aggregated IgM.

In a further aspect, the control stimulus is phosphate-buffered saline.

In a further aspect, at least one of the first, second, third, and fourth samples comprises whole blood.

In a further aspect, the significant difference in the ratios is that the first ratio is greater than the second ratio.

In a further aspect, the therapy comprises administration of an agent selected from the group consisting of cyclosporine A and tacrolimus.

In an embodiment, a method of identifying a putative agent for treating rheumatoid arthritis is provided that comprises: obtaining first, second, third, and fourth samples comprising leukocytes from a human whose leukocytes demonstrate at least a 1.5-fold increase in the transcription of an mRNA selected from the group consisting of tumor necrosis factor subfamily (“TNFSF”)-3, TNFSF-4, TNFSF-7, TNFSF-11, and TNFSF-14 when exposed to heat-aggregated human IgG; stimulating leukocytes in vitro in the first sample; stimulating leukocytes in vitro in the second sample with a control stimulus; measuring the amount of the mRNA in the first and second samples after stimulation; calculating a first ratio of the amount of the mRNA in the first sample to the amount of the mRNA in the second sample; exposing the third and fourth samples to the agent; stimulating leukocytes in vitro in the third sample after the exposure; stimulating leukocytes in vitro in the fourth sample with the control stimulus after the exposure; measuring the level of the mRNA in the third and fourth samples after stimulation; calculating a second ratio of the amount of the mRNA in the third sample to the amount of the mRNA in the fourth sample; and comparing the first and second ratios, wherein a significant difference in the ratios is indicative of a putative agent.

In a further aspect, stimulating leukocytes in the first and third samples comprises intermixing heat-aggregated human IgG with the samples.

In a further aspect, stimulating leukocytes in the first and third samples comprises intermixing with the sample an agent selected from the group consisting of phorbol myristate acetate (PMA), phytohemagglutinin (PHA), wheat germ agglutinin (WGA), concanavalin-A (ConA), lipopolysaccharides (LPS), jacalin, fucoidan, heat-aggregated IgE, heat-aggregated IgA, and heat-aggregated IgM.

In a further aspect, the control stimulus is phosphate-buffered saline.

In a further aspect, at least one of the first, second, third, and fourth samples comprises whole blood.

In a further aspect, the significant difference in the ratios is that the first ratio is greater than the second ratio.

In an embodiment, a method of evaluating the state of rheumatoid arthritis in a human is provided that comprises: stimulating leukocytes in vitro in a first sample that comprises leukocytes and is obtained at a first time from the human; after the stimulation, measuring the amount of an mRNA selected from the group consisting of tumor necrosis factor subfamily (“TNFSF”)-3, TNFSF-4, TNFSF-7, TNFSF-11, and TNFSF-14 in the first sample; stimulating leukocytes in vitro with a control stimulus in a second sample comprising leukocytes obtained from the human at the first time; measuring the amount of the mRNA in the second sample after stimulation; determining a first ratio of the amount of the mRNA in the first sample to the amount of the mRNA in the second sample; stimulating leukocytes in vitro in a third sample that comprises leukocytes and is obtained from the human at a second time that is subsequent to the first time; after the stimulation, measuring the amount of the mRNA in the third sample; stimulating leukocytes in vitro with a control stimulus in a fourth sample comprising leukocytes obtained from the human at the second time; measuring the amount of the mRNA in the fourth sample after stimulation; determining a second ratio of the amount of the mRNA in the third sample to the amount of the mRNA in the fourth sample; and comparing the first and second ratios, wherein a significant difference in the first and second ratios is indicative of a change in the disease state.

In a further aspect, stimulating leukocytes in the first and third samples comprises intermixing human heat-aggregated IgG with the sample.

In a further aspect, stimulating leukocytes in the first and third samples comprises intermixing with the sample an agent selected from the group consisting of phorbol myristate acetate (PMA), phytohemagglutinin (PHA), wheat germ agglutinin (WGA), concanavalin-A (ConA), lipopolysaccharides (LPS), jacalin, fucoidan, heat-aggregated IgE, heat-aggregated IgA, and heat-aggregated IgM.

In a further aspect, the control stimulus comprises phosphate-buffered saline.

In a further aspect, at least one of the first, second, third and fourth samples comprises whole blood.

In a further aspect, the significant difference in the ratios is that the second ratio is greater than the first ratio, and the change in disease state is a progression of the disease.

In a further aspect, the significant difference in the ratios is that the first ratio is greater than the second ratio, and the change in disease state is a regression of the disease.

### BRIEF DESCRIPTION OF THE DRAWINGS

FIG. 1 shows the results of a quantification of FcR-mediated gene expression of various TNFSF mRNAs in human leukocytes in peripheral whole blood of rheumatoid arthritis patients and controls.

FIG. 2 shows the fold increases in various TNFSF mRNA levels induced by heat aggregated IgG (HAG) stimulation in the whole blood of rheumatoid arthritis patients and control patients.

FIGS. 3A and 3B show the effect of HAG on TNFSF and chemokine mRNA expression in peripheral blood leukocytes.

FIG. 4 shows the results of stimulation of whole blood of healthy individuals with phorbol myristate acetate (PMA).

FIG. 5 shows the results of stimulation of whole blood of healthy individuals with phytohemagglutinin (PHA).

FIG. 6 shows the results of stimulation of whole blood of healthy individuals with wheat germ agglutinin (WGA)

FIG. 7 shows the results of stimulation of whole blood of healthy individuals with concanavalin-A (ConA).

FIG. 8 shows the results of stimulation of whole blood of healthy individuals with lipopolysaccharides (LPS).

FIG. 9 shows the results of stimulation of whole blood of healthy individuals with jacalin.

FIG. 10 shows the results of stimulation of whole blood of healthy individuals with fucoidan.

FIGS. 11A-11D show the results of stimulation of whole blood of healthy individuals with heat-aggregated IgG.

FIGS. 12A-12C show the results of stimulation of whole blood of healthy individuals with heat-aggregated IgA.

FIGS. 13A-13C show the results of stimulation of whole blood of healthy individuals with heat-aggregated IgM.

FIGS. 14A-14C shows the results of stimulation of whole blood of healthy individuals with heat-aggregated IgE.

### DETAILED DESCRIPTION OF THE PREFERRED EMBODIMENT

The present disclosure relates to the use of differential mRNA transcription patterns in leukocytes in response to specific cellular stimuli in assessing whether RA patients are good candidates for specific therapies. The present disclosure also relates to the use of such differential transcription patterns in assessing whether therapy administered to a RA patient is effective. The present disclosure also relates to the use of such differential transcription patterns in screening candidate agents for use in treating RA. The present disclosure also relates to the use of such differential transcription patterns in evaluating the state of RA in patients over time and monitoring the progression of the disease.

As described above, the pathology of RA may be related to the functioning of the FcR in the immune cells of a RA patient. In order to further assess the possible role of the FcR in the disease, it is useful to assess whether the function of the FcR in circulating leukocytes in peripheral blood is normal before the leukocytes migrate to the pathological sites in patients with autoimmune disease, or is already enhanced before this migration. In order to analyze whether the function of the Fc receptor (FcR) was normal or enhanced in peripheral blood leukocytes in patients with rheumatoid arthritis (RA), heat-aggregated human IgG (HAG) was added directly into heparinized whole blood, and the changes in the mRNA level of various members of the tumor necrosis factor super family (TNFSF) were assessed. Although multiple FcRs exist for IgG (FcγR), such as FcγRI, Ia, IIb, and III (GeneBank UniGene database), HAG acts as a universal stimulus that can react with all FcR subtypes. The changes in the mRNA level of members of the TNF superfamily (TNFSF) mRNA (see, for example, the GeneBank UniGene database) resulting from the stimulus with HAG were quantified.

The method employed was as follows. Nucleotide sequences for various TNFSF genes were retrieved from the UniGene database in the GenBank. PCR primers for each gene were designed by Primer Express (Applied Biosystem, Foster City, Calif.) and HYBsimulator (RNAture, Irvine, Calif.) (see Mitsuhashi et al., Nature 367:759 (1994), and Hyndman et al., BioTechniques, 20:1090 (1996)) The sequences are summarized in Table I below. Oligonucleotides were synthesized by IDT (Coralville, Iowa).

TABLE 1
Primer Sequences

| | | |
|---|---|---|
| Target mRNA | Forward | Reverse |

| | | |
|---|---|---|
| TNFSF- 1 | CAGCTATCCACCCACACAGATG | CGAAGGCTCCAAAGAAGACAGT |
| | (SEQ ID NO: 1) | (SEQ ID NO: 2) |
| TNFSF-2 | TCAATCGGCCCGACTATCTC | CAGGGCAATGATCCCAAAGT |
| | (SEQ ID NO: 3) | (SEQ ID NO: 4) |
| TNFSF-3 | AGGGTGTACGTCAACATCAGTCA | CACGGCCCCAAAGAAGGT |
| | (SEQ ID NO: 5) | (SEQ ID NO: 6) |
| TNFSF-4 | GCCCCTCTTCCAACTGAAGAA | GGTATTGTCAGTGGTCACATTCAAG |
| | (SEQ ID NO: 7) | (SEQ ID NO: 8) |
| TNFSF-5 | CCACAGTTCCGCCAAACCT | CACCTGGTTGCAATTCAAATACTC |
| | (SEQ ID NO: 9) | (SEQ ID NO: 10) |
| TNFSF-6 | TGGCAGCATCTTCACTTCTAAATG | GAAATGAGTCCCCAAAACATCTCT |
| | (SEQ ID NO: 11) | (SEQ ID NO: 12) |
| TNFSF-7 | CACACTCTGCACCAACCTCACT | TGCACTCCAAAGAAGGTCTCATC |
| | (SEQ ID NO: 13) | (SEQ ID NO: 14) |
| TNFSF-8 | ACCACCATATCAGTCAATGTGGAT | GAAGATGGACAACACATTCTCAAGA |
| | (SEQ ID NO: 15) | (SEQ ID NO: 16) |
| TNFSF-9 | AGCTACAAAGAGGACACGAAGGA | CGCAGCTCTAGTTGAAAGAAGACA |
| | (SEQ ID NO: 17) | (SEQ ID NO: 18) |
| TNFSF-10 | GGGAATATTTGAGCTTAAGGAAAATG | AAAAGGCCCCGAAAAAACTG |
| | (SEQ ID NO: 19) | (SEQ ID NO: 20) |
| TNFSF-11 | GAGTATCTTCAACTAATGGTGTACGTCACT | TGGTGCTTCCTCCTTTCATCA |
| | (SEQ ID NO: 21) | (SEQ ID NO: 22) |
| TNFSF-12 | TACTGTCAGGTGCACTTTGATGAG | GCAGTGGCTGAGAATTCCT |
| | (SEQ ID NO: 23) | (SEQ ID NO: 24) |
| TNFSF-13 | ATATGGTGTCCGAATCCAGGAT | CCTGACCCATGGTGAAAGTCA |
| | (SEQ ID NO: 25) | (SEQ ID NO: 26) |
| TNFSF-13B | ATGCCTGAAACACTACCCAATAATT | GCAAGTTGGAGTTCATCTCCTTCT |
| | (SEQ ID NO: 27) | (SEQ ID NO: 28) |
| TNFSF-14 | CGTCCGTGTGCTGGATGA | CATGAAAGCCCCGAAGTAAGAC |
| | (SEQ ID NO: 29) | (SEQ ID NO: 30) |
| TNFSF-15 | TGCGAAGTAGGTAGCAACTGGTT | CCATTAGCTTGTCCCCTTCTTG |
| | (SEQ ID NO: 31) | (SEQ ID NO: 32) |
| TNFSF-18 | CGGCTGTATAAAAACAAAGACATGAT | TCCCCAACATGCAATTCATAAG |
| | (SEQ ID NO: 33) | (SEQ ID NO: 34) |
| IL-1B | GAAGATGGAAAAGCGATTTGTCTT | GGGCATGTTTTCTGCTTGAGA |
| | (SEQ ID NO: 35) | (SEQ ID NO: 36) |
| IL-5 | GCTCTTGGAGCTGCCTACGT | AAGGTCTCTTTCACCAATGCACTT |
| | (SEQ ID NO: 37) | (SEQ ID NO: 38) |
| IL-6 | TCATCACTGGTCTTTTGGAGTTTG | TCTGCACAGCTCTGGCTTGT |
| | (SEQ ID NO: 39) | (SEQ ID NO: 40) |
| IL-8 | TGCTAAAGAACTTAGATGTCAGTGCAT | TGGTCCACTCTCAATCACTCTCA |
| | (SEQ ID NO: 41) | (SEQ ID NO: 42) |
| IL-12A | GCAGGCCCTGAATTTCAACA | GAAGTATGCAGAGCTTGATTTTAGTTTTA |
| | (SEQ ID NO: 43) | (SEQ ID NO: 44) |
| IL-12B | GAAGTATGCAGAGCTTGATTTTAGTTTTA | CCCATTCGCT CCAAGATGAG |
| | (SEQ ID NO: 45) | (SEQ ID NO: 46) |
| IL-15 | TGAAGTGCTTTCTCTTGGAGTTACA | CATTCCCATTAGAAGACAAACTGTTG |
| | (SEQ ID NO: 47) | (SEQ ID NO: 48) |
| IL-16C | AAAACCTCTTGGGAAGCATGAG | GGGACCCCGAGGACAGTACT |
| | (SEQ ID NO: 49) | (SEQ ID NO: 50) |
| CCL-2 | CCATTGTGGCCAAGGAGATC | TGTCCAGGTGGTCCATGGA |
| | (SEQ ID NO: 51) | (SEQ ID NO: 52) |
| CCL-3 | CACAGAATTTCATAGCTGACTACTTTGA | TCGCTTGGTTAGGAAGATGACA |
| | (SEQ ID NO: 53) | (SEQ ID NO: 54) |
| CCL-4 | GGTATTCCAAACCAAAAGAAGCA | GTTCAGTTCCAGGTCATACACGTACT |
| | (SEQ ID NO: 55) | (SEQ ED NO: 56) |
| CCL-5 | AGTCGTCTTTGTCACCCGAAA | AGCTCATCTCCAAAGAGTTGATGTAC |
| | (SEQ ID NO: 57) | (SEQ ID NO: 58) |
| CCL-7 | TGTGCTGACCCCACACAGA | GCTTTGGAGTTTGGGTTTTCTTG |
| | (SEQ ID NO: 59) | (SEQ ID NO: 60) |
| CCL-8 | AGAGCTACACAAGAATCACCAACATC | AGACCTCCTTGCCCCGTTT |
| | (SEQ ID NO: 61) | (SEQ ID NO: 62) |
| CCL-11 | CCCAGAAAGCTGTGATCTTCAA | TCCTGCACCCACTTCTTCTTG |
| | (SEQ ID NO: 63) | (SEQ ID NO: 64) |
| CCL-13 | CCAAACTGGGCAAGGAGATCT | GGCCCAGGTGTTTCATATAATTCT |
| | (SEQ ID NO: 65) | (SEQ ID NO: 66) |
| CCL-14 | TGCTTCACCTACACTACCTACAAGATC | GACAATTCCGGGCTTGGA |
| | (SEQ ID NO: 67) | (SEQ ID NO: 68) |
| CCL-18 | CAGATTCCACAAAAGTTCATAGTTGAC | CCGGCCTCTCTTGGTTAGG |
| | (SEQ ID NO: 69) | (SEQ ID NO: 70) |
| CCL-19 | CTGCTGTAGTGTTCACCACACTGA | CTGCTGTAGTGTTCACCACACTGA |
| | (SEQ ID NO: 71) | (SEQ ID NO: 72) |
| CCL-20 | GATACACAGACCGTATTCTTCATCCTAA | TGAAAGATGATAGCATTGATGTCACA |
| | (SEQ ID NO: 73) | (SEQ ID NO: 74) |
| CCL-21 | CGCTCTCAGGCAGAGCTATGT | CTTGTCCAGATGCTGCATCAG |
| | (SEQ ID NO: 75) | (SEQ ID NO: 76) |
| CCL-22 | GCGCGTGGTGAAACACTTC | ATCGGCACAGATCTCCTTATCC |
| | (SEQ ID NO: 77) | (SEQ ID NO: 78) |
| CCL-23 | CGAAGCATCCCGTGTTCACT | GATGACACCCGGCTTGGA |
| | (SEQ ID NO: 79) | (SEQ ID NO: 80) |
| CCL-24 | CAGGAGTGATCTTCACCACCAA | GGCGTCCAGGTTCTTCATGT |
| | (SEQ ID NO: 81) | (SEQ ID NO: 82) |
| CCL-25 | GGCGTCCAGGTTCTTCATGT | GTAGAATATCGCAGCAGGCAGAT |
| | (SEQ ID NO: 83) | (SEQ ID NO: 84) |
| CCL-26 | CTGCTTCCAATACAGCCACAAG | GAGCAGCTGTTACTGGTGAATTCA |
| | (SEQ ID NO: 85) | (SEQ ID NO: 86) |
| CCL-27 | CGTGCTTCACCTGGCTCAA | GGTGCTCAAACCACTGTGACA |
| | (SEQ ID NO: 87) | (SEQ ID NO: 88) |
| CCL-28 | GGAAATGTTTGCCACAGGAAGA | TGTTTCGTGTTTCCCCTGATG |
| | (SEQ ID NO: 89) | (SEQ ID NO: 90) |
| CXCL-1 | CCACTGCGCCCAAACC | GCAGGATTGAGGCAAGCTTT |
| | (SEQ ID NO: 91) | (SEQ ID NO: 92) |
| CXCL-2 | CCCCTGGCCACTGAACTG | TGGATGTTCTTGAGGTGAATTCC |
| | (SEQ ID NO: 93) | (SEQ ID NO: 94) |
| CXCL-3 | GGAATTCACCTCAAGAACATCCA | GTGGCTATGACTTCGGTTTGG |
| | (SEQ ID NO: 95) | (SEQ ID NO: 96) |
| CXCL-4 | CCGTCCCAGGCACATCAC | CCGTCCCAGGCACATCAC |
| | (SEQ ID NO: 97) | (SEQ ID NO: 98) |
| CXCL-5 | AGAGCTGCGTTGCGTTTGT | TGGCGAACACTTGCAGATTACT |
| | (SEQ ID NO: 99) | (SEQ ID NO: 100) |
| CXCL-6 | CAGAGCTGCGTTGCACTTGT | ACACCTGCAGTTTACCAATCGTT |
| | (SEQ ID NO: 101) | (SEQ ID NO: 102) |
| CXCL-7 | TCTGGAATTCATCCCAAAAACA | TCTGGAATTCATCCCAAAAACA |
| | (SEQ ID NO: 103) | (SEQ ID NO: 104) |
| CXCL-9 | CCACCTACAATCCTTGAAAGACCTT | CAGTGTAGCAATGATTTCAATTTTCTC |
| | (SEQ ID NO: 105) | (SEQ ID NO: 106) |
| CXCL-10 | TCCACGTGTTGAGATCATTGC | TCTTGATGGCCTTCGATTCTG |
| | (SEQ ID NO: 107) | (SEQ ID NO: 108) |
| CXCL-16 | CCCACAGCCAGGAGATCAG | CTTGCACAGCACATAGGAAAGG |
| | (SEQ ID NO: 109) | (SEQ ID NO: 110) |

Heat aggregated IgG (HAG) was prepared by heating 20 mg/mL human IgG (Sigma, St. Louis) in PBS at 63° C. for 15 min (see Ostreiko et al., Immunol Lett. 15, 311 (1987)). In 8-well strip microtubes, 1.2 μl of HAG or control (phosphate buffered saline) (BioLegend, San Diego) were added, and stored at −20° C. until use. Sixty μl of fresh heparinized whole blood was added into each well in triplicate, and incubated at 37° C. for 2-8 hours with cap closed. After each treatment, 50 μl of whole blood was transferred to filterplates as described below. Each blood sample was stored frozen at −80° C. until use.

The mRNA and cDNA were prepared from whole blood following the method set forth in Mitsuhashi et al., Clin. Chem. 52:4 (published as doi:10.1373/clinchem. 2005.048983). The method disclosed in U.S. patent application Ser. No. 10/796,298, which is incorporated here by reference, may also be employed. In brief, 96-well filterplates were placed over collection plates, and 150 μl 5 mM Tris, pH 7.4, was applied. Following centrifugation at 120×g for 1 min at 4° C., 50 Ml of blood sample was applied to each well and immediately centrifuged at 120×g for 2 min at 4° C., followed by washing of each well with 300 μl PBS once with centrifugation at 2000×g for 5 min at 4° C. Then, 60 μl stock lysis buffer, supplemented with 1% 2-mercaptethanol (Bio Rad, Hercules, Calif., USA), 0.5 mg/ml proteinase K (Pierce, Rockford, Ill., USA), 0.1 mg/ml salmon sperm DNA (5 Prime EppendorfiBrinkmann, Westbury, N.Y., USA), 0.1 mg/ml E. coli tRNA (Sigma), a cocktail of 10 mM each of specific reverse primers, and standard RNA34 oligonucleotides, were applied to the filterplates, followed by incubation at 37° C. for 10 min. The filterplates were then placed over oligo(dT)-immobilized microplates (GenePlate, RNAture) (see Mitsuhashi et al., Nature 357:519 (1992), and Hamaguchi et al., Clin. Chem. 44, 2256 (1998), both incorporated herein by reference), and centrifuged at 2000×g for 5 min at 4° C. Following overnight storage at 4° C., the microplates were washed with 100 μl plain lysis buffer 3 times, followed by 150 μl wash buffer (0.5 M NaCl, 10 mM Tris, pH 7.4, 1 mM EDTA) 3 times at 4° C. The cDNA was directly synthesized in each well by adding 30 μl buffer containing 1×RT-buffer, 1.25 mM each of dNTP, 4 units rRNasin, and 80 units of MMLV reverse transcriptase (Promega) (without primers), and incubation at 37° C. for 2 hours. The specific primer-primed cDNA existed in solution, and oligo(dT)-primed cDNA stayed immobilized in the microplate (see Hugli, Crit. Rev. Immunol. 1, 321 (1981)). For SYBR Green PCR (see Morrison et al., Biotechniques 24, 954 (1998), incorporated herein by reference), cDNA was diluted 4-fold in water, and 4 μl of cDNA solution was directly transferred to 384-well PCR plates, to which 5 μl iTaq SYBR master mix (BioRad, Hercules, Calif.) and 1 μl oligonucleotide cocktail (15 μM each of forward and reverse primer) were applied, and PCR was conducted in PRISM 7900HT (ABI), with one cycle of 95° C. for 10 min followed by 45 cycles of 95° C. for 30 sec and 60° C. for 1 min. TaqMan PCR could also be employed; in such a case, the cDNA solution is directly transferred to 384-well PCR plates, to which 5 μl of TaqMan universal master mix (ABI) and 1 μl oligonucleotide cocktail (15 μM each of forward and reverse primer, and 3-6 μM TaqMan probe) are applied, and PCR is conducted in PRISM 7900HT (ABI), with one cycle of 95° C. for 10 min followed by 45 cycles of 95° C. for 30 sec, 55° C. for 30 sec, and 60-65° C. for 1 min.

The 1×RT buffer was used as a negative control to confirm that no primer dimer was generated under SYBR Green PCR conditions. The Ct was determined by analytical software (SDS, ABI). The ΔCt was calculated by subtracting the Ct values of appropriate control samples, and the fold increase was calculated as 2̂(−ΔCt), by assuming that the efficiency of each PCR cycle was 100%.

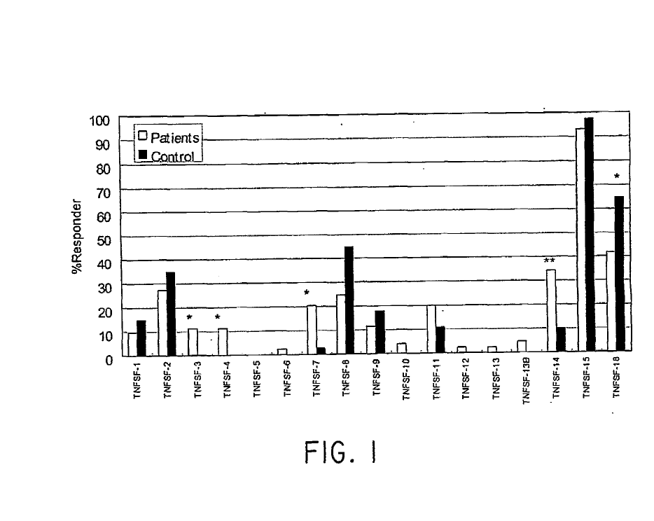

FIG. 1 shows the results of an analysis of the FcR-mediated gene expression of TNFSF mRNA in human leukocytes in peripheral whole blood. The results shown for R^{A} patients are expressed as a percentage of “responder” subjects, defined as those subjects that exhibit a fold increase of greater than 1.5 in response to HAG stimulation.

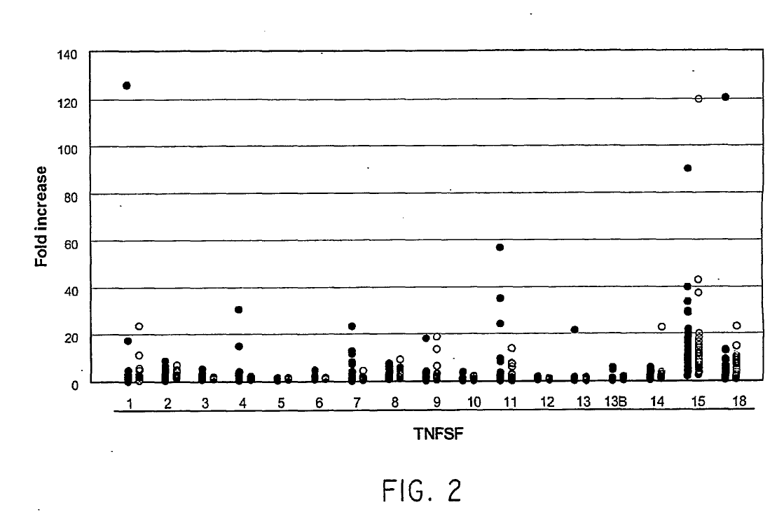

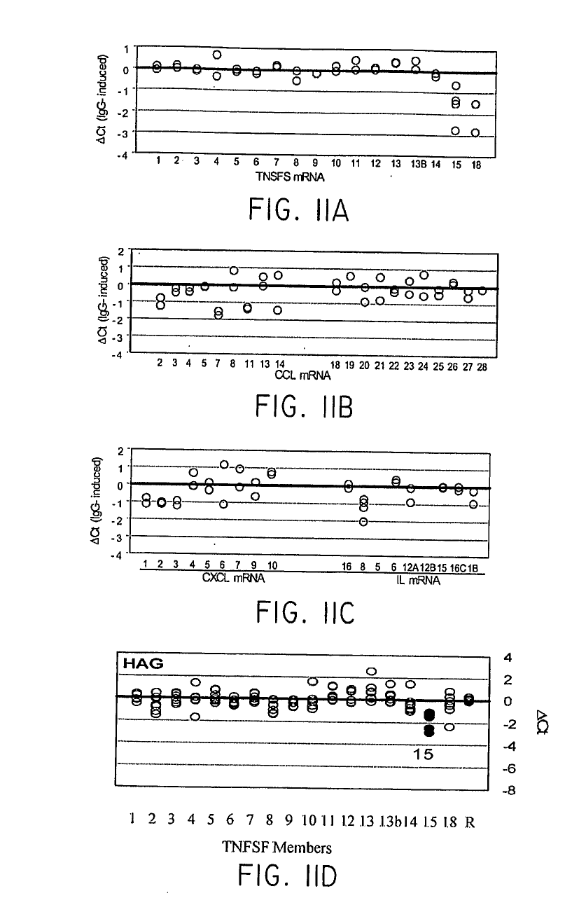

The individual results for RA patients and healthy control subjects are expressed in FIG. 2 as a fold increase over the control values. Each datum (o for healthy adults and for RA patients) was the mean from triplicate aliquots of whole blood. The statistical significance shown in FIG. 1 (*: p<0.05, **: p<0.01) was calculated by χ² test using the population of responders and non-responders, as described above. Further data obtained using whole blood of healthy individuals stimulated with HAG and expressed in terms of the cycle threshold (Ct) (see below), are shown in FIG. 11.

As shown in FIGS. 1 and 2, HAG mainly induced TNFSF-3, 4, 7, 8, 11, 14, 15, and 18 mRNAs. The responder population (>1.5 fold increase) for HAG-induced TNFSF-3, 4, 7, and 14 was significantly larger in RA patients than in healthy controls, and the responder population for HAG-induced TNFSF-11 was larger in HAG than in controls, although statistical significance could not be established in this case. These data suggest impairment of HcR function in peripheral blood leukocytes, with attendant hyperfunction of leukocytes with respect to TNFSF-3, 4, 7, 11, and 14. This system will be useful in the analysis of cytotoxic functions of immune cells in RA, as described below.

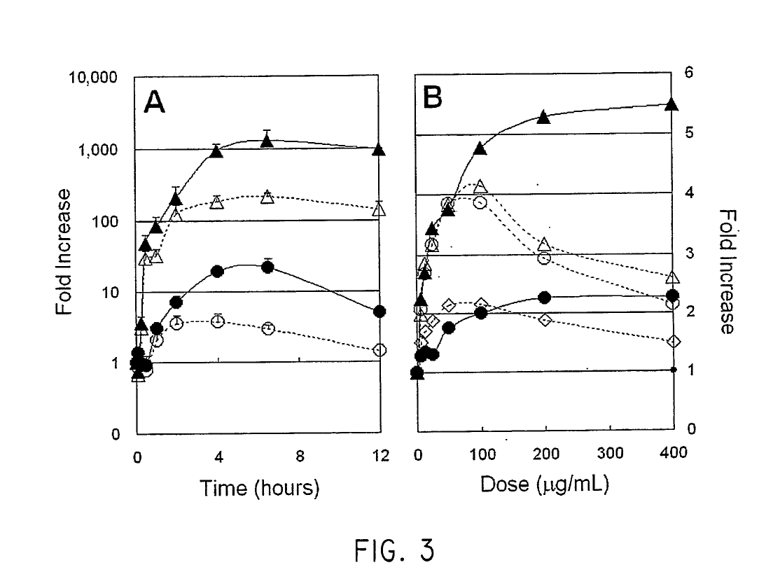

Dose responses and kinetic studies are shown in FIG. 3. FIG. 3 shows the effect of heat aggregated IgG (HAG) on TNFSF mRNA expression in peripheral blood leukocytes. FIG. 3A shows the kinetics of the reaction. Triplicate aliquots of 60 μl each of heparinized whole blood was mixed with PBS (◯, Δ), or 200 μg/mL HAG (, ▴) and incubated at 37° C. for 0-12 hours. TNFSF-15 (◯, ) and IL-8 (Δ, ▴) mRNA were then quantified as described above. The fold increase was calculated using the values at time=0 as a control. FIG. 3B shows the dose response. Triplicate aliquots of 60 μl each of heparinized whole blood were mixed with various concentrations of HAG and incubated at 37° C. for 2 hours. TNFSF-2 (◯), TNFSF-15 (▴), IL-8 (◯), IL-1B (⋄), and CXCL-2 (Δ) mRNA were then quantified as described above. The fold increase was calculated using the values for the solvent (PBS) as a control. Each data point was the mean±standard deviation (A) or mean (B) from triplicate aliquots of whole blood. External control RNA34 was unchanged in all cases, suggesting that the assay was performed appropriately.

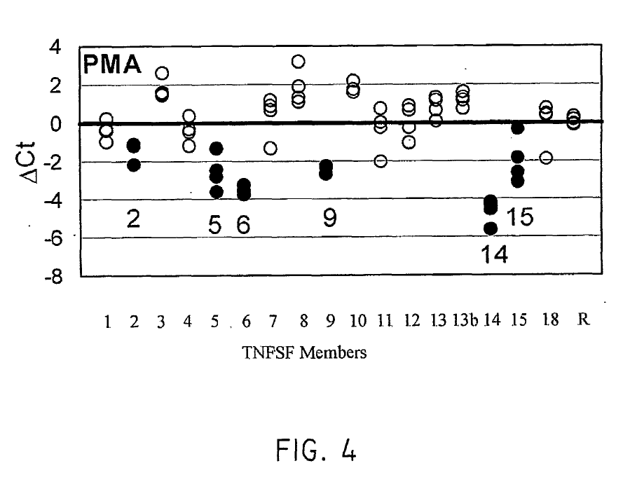

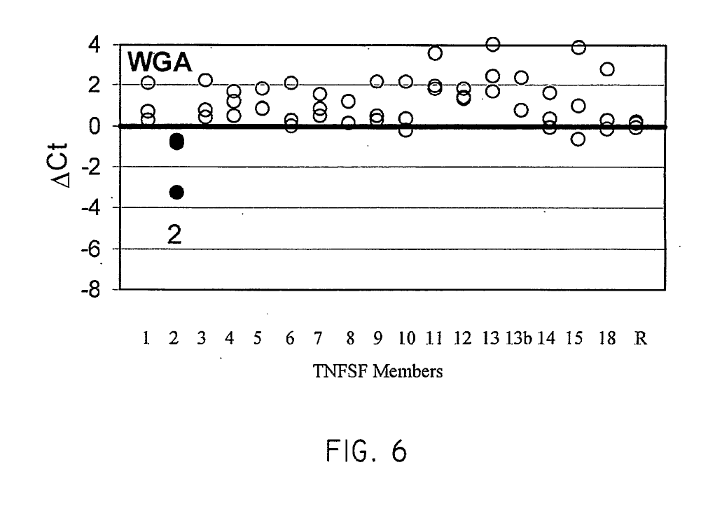

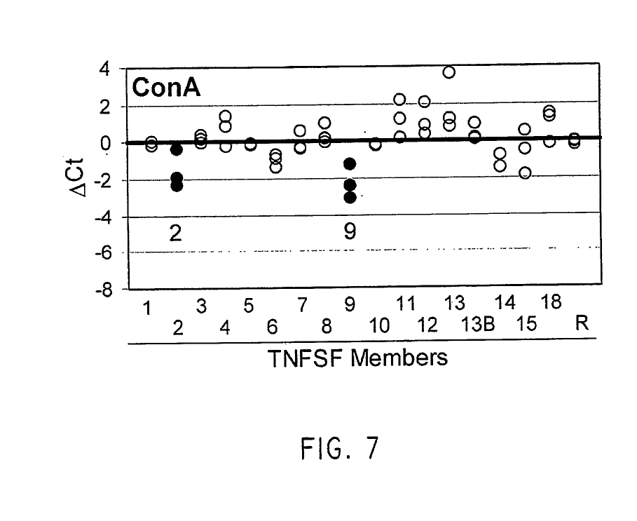

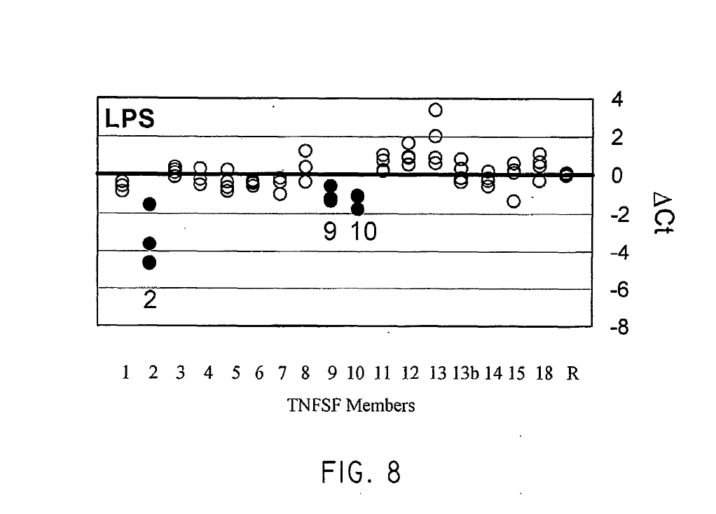

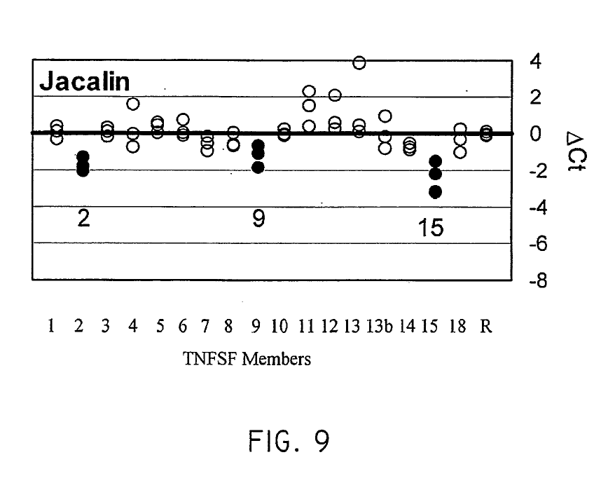

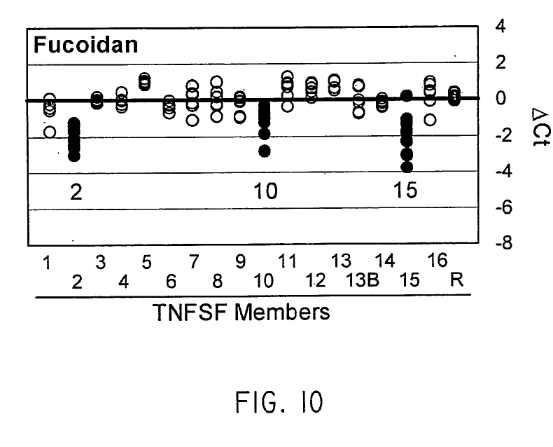

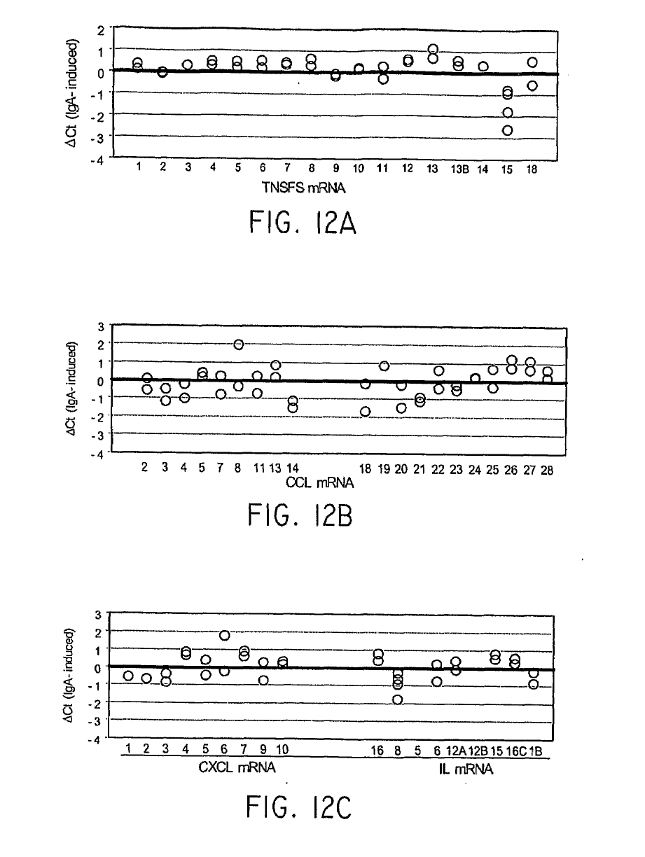

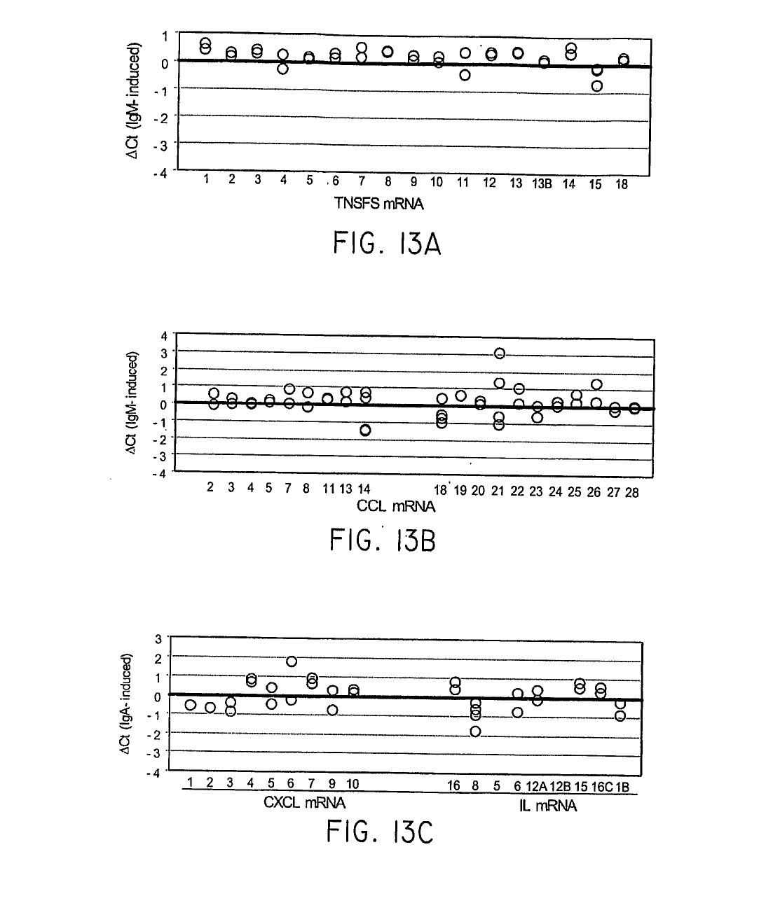

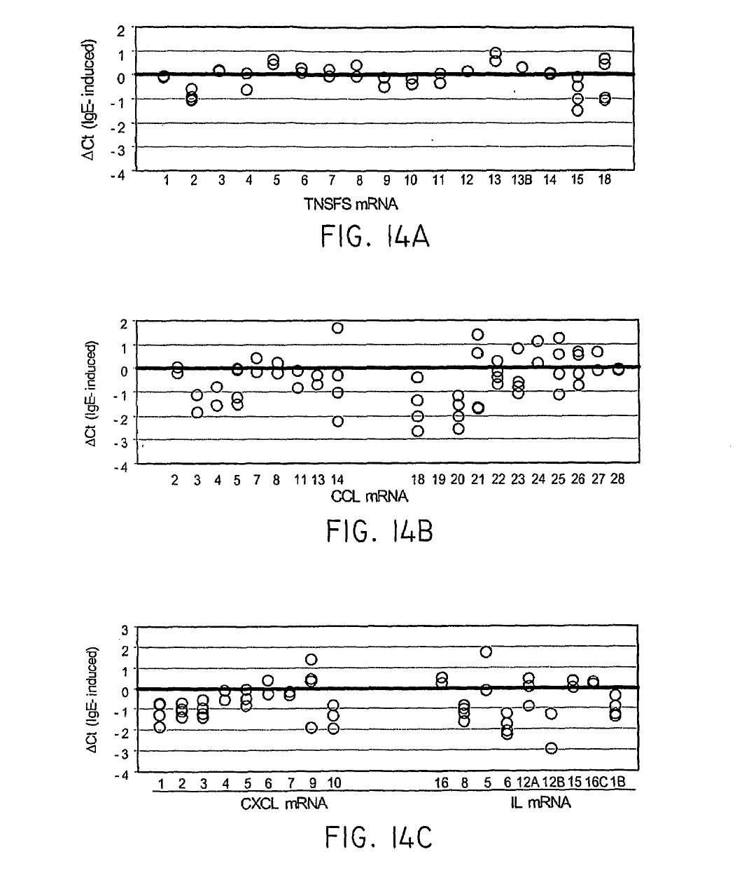

In addition to the HAG described above, other stimulating agents, such as phorbol myristate acetate (PMA), phytohemagglutinin (PHA), wheat germ agglutinin (WGA), concanavalin-A (Con-A), lipopolysaccharides (LPS), jacalin, fucoidan, heat-aggregated IgA, heat-aggregated IgE, and heat-aggregated IgM, also induce different subtypes of TNFSF and chemokines in whole blood taken from healthy individuals, as shown in FIGS. 4-10 and 12-14. The protocol followed in these assays was the same as that given above, with the exception of the different stimulus employed in each case. In FIGS. 4-14, data are expressed in terms of the cycle threshold (Ct), which is the number of cycles of PCR required to generate certain amounts of PCR products. The ACt values were obtained by subtracting Ct values of un-stimulated samples from stimulated samples. Since Ct is a log scale, 1 ΔCt unit indicates a change in quantity by a factor of 2. Because a higher expression level reduces the number of PCR cycles required to generate a standard amount of products, a negative ΔCt value indicates an increase in expression.

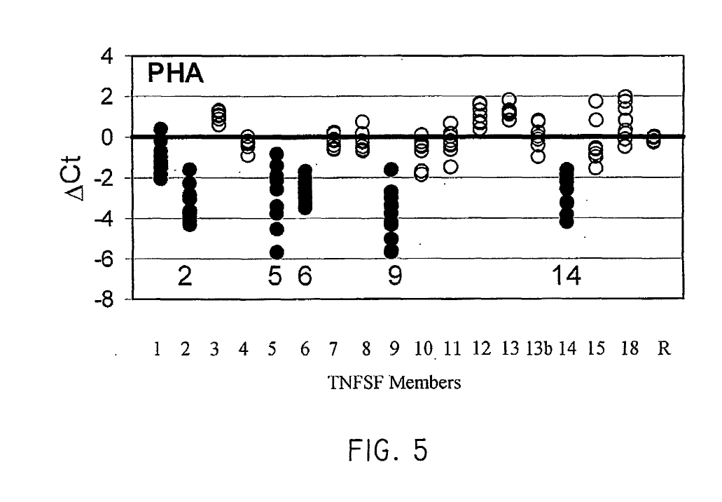

Some of these agents exhibit a stimulus pattern similar to that of HAG. In particular, as shown in FIGS. 4 and 5, PMA and PHA stimulate TNFSF-14, which is also stimulated in RA patients by HAG. These agents will also be useful in assessing the therapeutic options for RA patients and in screening for new drugs for treating RA.

Cytotoxic assays have generally been used to study actual cell death resulting from the activity of the immune system, such as that which is believed to occur in RA. Cytotoxic assays are generally conducted by incubating ⁵¹Cr-loaded target cells with effector cells at various ratios, and quantifying the amounts of ⁵¹Cr radioactivity released from the dead or damaged cells (see Dunkley et al., J Immunol Methods 6, 39 (1974)). Radioactive materials have been replaced with non-radioactive materials, such as fluorometric materials, in some cases (see Kruger-Krasagakes et al., J Inimunol Methods 156, 1 (1992)), but the basic principle is unchanged. The results of cytotoxic assays are thus reflective of actual cell death.

However, cytotoxic assays are performed under non-physiological experimental conditions, and complex cell-to-cell and cell-to-plasma interactions are difficult to assess in the course of such studies. Furthermore, cytotoxic assays do not indicate which TNFSF member is responsible for cell death. Once effector cells recognize the target cells, the effector cells' function is not only to kill the target, but also to recruit other effector cells, because a single effector cell is not enough to kill many target cells. This recruitment function is thought to be represented by the release of chemotactic factors. The identity of such chemotactic factors released by effector cells would not be revealed by classic cytotoxic assays. The assay system set forth in this disclosure is, however, capable of identifying many classes of gene expression in effector cells simultaneously.

Identification of responsible TNFSF subtypes is critically important, because these molecules react with specific receptors on the target cells or leukocytes. For example, according to UniGene's Expression Sequence Tag (EST) profile database, the receptor for TNFSF-11 (TNFRSF11, also known as RANK) is present on osteoclasts, and stimulation of this pathway leads to bone resorption. Thus, enhanced TNFSF-11 activity in RA patients (FIG. 1, FIG. 2) may be linked to bone erosion, which is a major effect of RA.

The use of whole blood is preferable to using isolated leukocytes in culture media, because the former is more physiological than the latter, and whole populations of leukocytes can be screened. Longer incubation of whole blood may produce additional artifacts. Thus, the ideal way is to identify early signals of killer and recruitment signals in whole blood during a short period of incubation by switching in vitro to ex vivo. The transcription of mRNA is an earlier event than either protein synthesis or the final biological outcomes. Thus, mRNA is a logical target.

The present disclosure is the first to suggest an underlying hyperfunction of TNFSF-11 inducibility from FcR on circulating leukocytes in peripheral blood. Since the present method uses whole blood, not intestinal tissues, it may be used as a diagnostic test for RA to evaluate possible responsiveness to TNFSF-inactivating therapy, and to monitor the therapeutic response. Specifically, in a preferred embodiment of a method for determining whether a human having RA is likely to respond to a therapy targeting an mRNA transcribed in response to T cell stimulation, whole blood is obtained from an RA patient and samples of the blood are subjected to HAG stimulation and optionally to control stimulation (PBS), as described above. The amount of mRNA level of TNFSF-3, TNFSF4, TNFSF-7, TNFSF-11, or TNFSF-14 mRNA may be measured in the samples as described above. An RA patient having a significantly elevated level of one or more of these mRNAs after stimulation with HAG (as indicated, for example, by a fold change of greater than 1.5) is a good candidate for therapy targeting these mRNAs.

Furthermore, in a preferred embodiment of a method of evaluating the effectiveness of RA treatment targeting one or more of the TNFSF-3, TNFSF-4, TNFSF-7, TNFSF-11, or TNFSF-14 mRNAs in a patient, a first ratio of the amount of the mRNA in whole blood after HAG stimulation to the amount after control stimulation is obtained prior to the initiation of the treatment. A second ratio of the amount of the mRNA in whole blood after HAG stimulation to the amount after control stimulation is obtained after the initiation of the treatment. A significant difference in the ratios, such as where the first ratio is larger than the second ratio, can indicate the effectiveness of the therapy. Such therapies could include, for example, the administration of cyclosporine A or tacrolimus.

Importantly, this ex vivo method can be used for the screening of compounds which inhibit anti-FcR-mediated expression of one or more of the TNFSF-3, TNFSF-4, TNFSF-7, TNFSF-11, or TNFSF-14 mRNAs, and particularly TNFSF-11, which is known to participate in osteoclast activation. Such compounds will be interesting drug targets, because these new drug candidates will block mRNA production in leukocytes at the transcription level. This will provide a new strategy for drug development against RA.

In an embodiment of a method of screening drug compounds using the disclosed system and thereby identifying a putative agent for treating RA, whole blood is obtained from RA patients that are responders, in that their leukocytes exhibit at least a 1.5-fold increase in the level of a RA-associated mRNA when exposed to a T-cell stimulation such as HAG. A first ratio of the amount of the mRNA in whole blood of the subjects after HAG stimulation to the amount after control stimulation is calculated. Further whole blood samples from the subjects are exposed in vitro to the drug compound, and then differentially stimulated as described above. A second ratio of the amount of the mRNA in whole blood after HAG stimulation to the amount after control stimulation of these exposed samples is then calculated. A significant difference in the two ratios, such as where the first ratio is larger than the second ratio, can indicate that the drug compound is a candidate for further investigation as a potential therapeutic for RA.

Additionally, in a preferred embodiment of a method of monitoring the state of the disease in a RA patient by measuring levels of one or more of TNFSF-3, TNFSF4, TNFSF-7, TNFSF-11, or TNFSF-14 mRNAs in samples comprising leukocytes obtained from the patient, a first ratio of the amount of the mRNA in whole blood after T-cell stimulus using heat-aggregated IgG antibody or other stimulus in vitro to the amount after control stimulation in vitro is obtained at a first time. At a second time subsequent to the first time, a second ratio of the amount of the mRNA in whole blood after the T-cell stimulus in vitro to the amount after control stimulation in vitro is obtained. A significant difference in the ratios can indicate a change in the disease state. For example, when the second ratio is larger than the first, this can indicate disease progression, while a larger first ratio can indicate that the disease has regressed.

1. A method of determining whether a human having rheumatoid arthritis is likely to respond to a therapy, comprising:
stimulating leukocytes in vitro in a first sample that comprises leukocytes from the human;
after the stimulation, measuring the amount of an mRNA selected from the group consisting of tumor necrosis factor superfamily (“TNFSF”)-3, TNFSF-4, TNFSF-7, TNFSF-11, and TNFSF-14 in the first sample;
stimulating leukocytes in vitro in a second sample comprising leukocytes from the human with a control stimulus;
measuring the amount of the mRNA in the second sample; and
determining a ratio of the amount of the mRNA in the first sample to the amount of the mRNA in the second sample, wherein the human is likely to respond to the therapy if the ratio is about 1.5:1 or greater.

2. The method of claim 1, wherein stimulating leukocytes in the first sample comprises intermixing heat-aggregated human IgG with the first sample.

3. The method of claim 1, wherein stimulating leukocytes in the first sample comprises intermixing with the first sample an agent selected from the group consisting of phorbol myristate acetate {PM A), phytohemagglutinin (PHA), wheat germ agglutinin (WGA), concanavalin-A (ConA), lipopolysaccharides (LPS), jacalin, fucoidan, heat-aggregated IgE, heat-aggregated IgA, heat-aggregated IgG, and heat-aggregated IgM.

4. The method of claim 1, wherein at least one of the first and second samples comprises whole blood.

5. The method of claim 1, wherein the control stimulus is phosphate buffered saline.

6. The method of claim 1, wherein the therapy comprises administration of an agent selected from the group consisting of cyclosporine A and tacrolimus.

7. A method of evaluating the effectiveness of a therapy for rheumatoid arthritis, comprising:
stimulating leukocytes in vitro in a first sample comprising leukocytes from the human;
stimulating leukocytes in vitro in a second sample comprising leukocytes from the human with a control stimulus;
measuring the amount of an mRNA selected from the group consisting of tumor necrosis factor superfamily (“TNFSF”)-3, TNFSF-4, TNFSF-7, TNFSF-11, and TNFSF-14 in the first and second samples after stimulation;
calculating a first ratio of the amount of the mRNA in the first sample to the amount of the mRNA in the second sample;
administering the therapy to the human;
stimulating leukocytes in vitro in a third sample comprising leukocytes from the human obtained after the administration of therapy;
stimulating leukocytes in vitro in a fourth sample comprising leukocytes from the human obtained after the administration of therapy with the control stimulus; measuring the level of the mRNA in the third and fourth samples after stimulation;
calculating a second ratio of the amount of the mRNA in the third sample to the amount of the mRNA in the fourth sample; and
comparing the first and second ratios, wherein a significant difference in the ratios is indicative of an effective therapy.

8. The method of claim 7, wherein stimulating leukocytes in the first and third samples comprises intermixing heat-aggregated human IgG with the sample.

9. The method of claim 7, wherein stimulating leukocytes in the first and third samples comprises intermixing with the sample an agent selected from the group consisting of phorbol myristate acetate (PMA), phytohemagglutinin (PHA), wheat germ agglutinin (WGA), concanavalin-A (ConA), lipopolysaccharides (LPS), jacalin, fucoidan, heat-aggregated JgE, heat-aggregated IgA, heat-aggregated IgG, and heat-aggregated IgM.

10. The method of claim 7, wherein the control stimulus is phosphate-buffered saline.

11. The method of claim 7, wherein at least one of the first, second, third, and fourth samples comprises whole blood.

12. The method of claim 7, wherein the significant difference in the ratios is that the first ratio is greater than the second ratio.

13. The method of claim 12, wherein the therapy comprises administration of an agent selected from the group consisting of cyclosporine A and tacrolimus.

14. A method of identifying a putative agent for treating rheumatoid arthritis, comprising:
obtaining first, second, third, and fourth samples comprising leukocytes from a human whose leukocytes demonstrate at least a I.S-fold increase in the transcription of an mRNA selected from the group consisting of tumor necrosis factor superfamily (“TNFSF”)-3, TNFSF-4, TNFSF-7, TNFSF-11, and TNFSF-14 when exposed to heat-aggregated human IgG;
stimulating leukocytes in vitro in the first sample;
stimulating leukocytes in vitro in the second sample with a control stimulus;
measuring the amount of the mRNA in the first and second samples after stimulation;
calculating a first ratio of the amount of the mRNA in the first sample to the amount of the mRNA in the second sample;
exposing the third and fourth samples to the agent;
stimulating leukocytes in vitro in the third sample after the exposure; stimulating leukocytes in vitro in the fourth sample with the control stimulus after the exposure;
measuring the level of the mRNA in the third and fourth samples after stimulation;
calculating a second ratio of the amount of the mRNA in the third sample to the amount of the mRNA in the fourth sample; and
comparing the first and second ratios, wherein a significant difference in the ratios is indicative of a putative agent.

15. The method of claim 14, wherein stimulating leukocytes in the first and third samples comprises intermixing heat-aggregated human IgG with the samples.

16. The method of claim 14, wherein stimulating leukocytes in the first and third samples comprises intermixing with the sample an agent selected from the group consisting of phorbol myristate acetate (PMA), phytohemagglutinin (PHA). wheat germ agglutinin (WGA), concanavalin-A (ConA), lipopolysaccharides (LPS), jacalin, fucoidan, heat-aggregated IgE, heat-aggregated IgA, heat-aggregated IgG, and heat-aggregated IgM.

17. The method of claim 14, wherein the control stimulus is phosphate-buffered saline.

18. The method of claim 14, wherein at least one of the first, second, third, and fourth samples comprises whole blood.

19. The method of claim 14, wherein the significant difference in the ratios is that the first ratio is greater than the second ratio.

20. A method of evaluating the state of rheumatoid arthritis in a human, comprising:
stimulating leukocytes in vitro in a first sample that comprises leukocytes and is obtained at a first time from the human;
after the stimulation, measuring the amount of an mRNA selected from the group consisting of tumor necrosis factor subfamily superfamily (“TNFSF”)-3, TNFSF-4, TNFSF-7, TNFSF-11, and TNFSF-14 in the first sample;
stimulating leukocytes in vitro with a control stimulus m a second sample comprising leukocytes obtained from the human at the first time;
measuring the amount of the mRNA in the second sample after stimulation;
determining a first ratio of the amount of the mRNA in the first sample to the amount of the mRNA in the second sample;
stimulating leukocytes in vitro in a third sample that comprises leukocytes and is obtained from the human at a second time that is subsequent to the first time;
after the stimulation, measuring the amount of the mRNA in the third sample;
stimulating leukocytes in vitro with a control stimulus in a fourth sample comprising leukocytes obtained from the human at the second time;
measuring the amount of the mRNA in the fourth sample after stimulation;
determining a second ratio of the amount of the mRNA in the third sample to the amount of the mRNA in the fourth sample; and
comparing the first and second ratios, wherein a significant difference in the first and second ratios is indicative of a change in the disease state.

21. The method of claim 20, wherein stimulating leukocytes in the first and third samples comprises intermixing human heat-aggregated IgG with the sample.

22. The method of claim 40, wherein stimulating leukocytes in the first and third samples comprises intermixing with the sample an agent selected from the group consisting of phorbol myristate acetate (PMA), phytohemagglutinin (PHA), wheat genn agglutinin (WGA), concanavalin-A (ConA), lipopolysaccharides (LPS), jacalin, fucoidan, heat-aggregated IgE, heat-aggregated IgA, heat-aggregated IgG, and heat-aggregated IgM.

23. The method of claim 20, wherein the control stimulus is phosphate-buffered saline.

24. The method of claim 20, wherein at least one of the first, second, third and fourth samples comprises whole blood.

25. The method of claim 20, wherein the significant difference in the ratios is that the second ratio is greater than the first ratio, and the change in disease state is a progression of the disease.

26. The method of claim 20, wherein the significant difference in the ratios is that the first ratio is greater than the second ratio, and the change in disease state is a regression of the disease.
